(19) United States
(12) Patent Application Publication
(10) Pub. No.: US 2007/0289070 A1
(43) Pub. Date: Dec. 20, 2007

(54) Additive For Domestic Washing Processes

(76) Inventors: Rafael Louzan Garcia, Barcelona (ES); Simon Velarde Esquina, Barcelona (ES)

Correspondence Address: PATENT, COPYRIGHT & TRADEMARK LAW GROUP, PO BOX 506, RICHFIELD, OH, 44286, US

(21) Appl. No.: 11/667,871

(22) PCT Filed: Nov. 16, 2005
(86) PCT No.: PCT/EP05/12300

§ 371 Date: May 16, 2007

(30) Foreign Application Priority Data
Nov. 23, 2004 (ES) P 200402820

(51) Int. Cl. C11D 17/00; A61K 8/11; A61K 9/16; D06M 23/12; C11D 3/50; A61K 9/50

(52) U.S. Cl. 8/137; 252/8.91

(57) ABSTRACT

Additive for domestic washing processes, for incorporation in the process after washing and rinsing, which is comprised of a microencapsulated product which is either a cosmetic, a pharmacological drug, or an evaporable product, the microcapsule coating being comprised of a cationic compound. The additive is preferably in the form of a liquid and in unit doses. The invention also relates to a process for treating fibres with the additive.

### TECHNICAL FIELD OF THE INVENTION

The present invention relates to an additive for domestic washing processes. It also relates to a process for treating fibres in a domestic washing process.

### BACKGROUND OF THE INVENTION

In domestic washing processes, after washing with detergent to remove dirt from the clothing, the clothes are usually rinsed with products for special treatment, called products for the finishing treatment of washed clothes. For example, such as special treatment products usually used with fabric softeners which in turn incorporate some perfumed substance. However, in the case of perfumed softeners, this finishing cycle does not, on many occasions, have a long-lasting effect, with the washed clothes very quickly ceasing to emit the perfume they were impregnated with.

With the aim of prolonging the emission of perfume in fabric softeners, the perfume is contained in said softener in the form of microcapsules whose nucleus contains the desired perfume. An example of this type of fabric softener is disclosed in patent ES 2063241, wherein microcapsules and the fabric softeners containing them are described, to improve the finishing cycle of the clothing and prolonging their scent and feeling of freshness. Also, in WO 0162376, micro and nanocapsules are disclosed which are incorporated into detergents and cleaning products, for example, for the treatment of fabrics, such as mild detergents, stain removers, universal detergents, etc.

The microcapsules, or micrometric-sized capsules comprised of a nucleus covered in a coating suitable for holding the nucleus inside, are being used in many fields of the art. One of these is the finishing treatment of textiles or fibrous materials to give the fibres the required properties, for example, to make ironing easier, to make the fibres fire-resistant or water-proof, or to release a perfume or cosmetic agent of interest.

In this latter case, garments can be found on the market that incorporate a microencapsulated moisturizing and/or tonic agent, such as, for example, underwear with a microencapsulated firming agent which is released by the effect of rubbing against the wearer's skin and which comes into contact with the skin, being absorbed and, hence, releasing its effect. The use of microcapsules is done at manufacturing level, generally before the garments, that are to incorporate the microcapsules with the agent of interest, are made. An example of this is disclosed WO 03026594, wherein ginger oil is encapsulated in microcapsules which are applied to fabrics, more specifically in the manufacture of tights and/or socks, the ginger oil being released when it rubs against the skin, which enables improved microcirculation and reduces swelling of the lower extremities.

The problem with said garments is that once the microencapsulated active agent has been completely released, the garments or fibrous materials become conventional garments or fibres without the desired effects, because the microcapsules have opened and the reserves of the agent of interest used up, or because said agent has diffused through the barrier or coating of the microcapsules.

In an attempt to provide and prolong the emission of a determined perfume, U.S. Pat. No. 4,234,627 discloses a composition for the process of domestic prewashing or washing which comprises a granular textile treatment mixture and an adjuvant for pre-washing or washing. The granular mixture contains some microcapsules and a so-called microcapsule transfer agent, which in practice refers to an organic material of the type conventionally used as domestic-use softening agents. However, in this case, the granular component with microcapsules is suitable for the pre-washing and washing process and subsequent drying of the clothes, incorporating a detergent or surfactant which is soluble in water. Said composition is sufficient for perfuming the garments that are washed, but it is a complex composition which has the added drawback that the user has to use the detergent or softener accompanying the granular mix.

The additive object of the present invention provides new solutions to said problems, while offering notable advantages to the state of the art.

### EXPLANATION OF THE INVENTION

The additive for domestic washing processes, for incorporation in the process after washing and rinsing, is characterized in that it is comprised of a microencapsulated product which is either a cosmetic, a pharmacological drug, an evaporable product or a combination thereof, the microcapsule coating being comprised of a cationic compound.

According to another characteristic of the invention, the additive is in the form of a monodose.

The additive according to the invention is also characterized in that it is in liquid form.

According to another characteristic of the additive of the invention, the cosmetic product is selected independently from among a moisturizing compound, an ultra-violet ray protector, a toner, a product for reducing the effects of stress on the skin, an anti-wrinkle compound, a deodorant, a compound to slow hair-growth and/or a whitening compound.

The additive according to the invention comprises a pharmacological product which is selected independently from among a painkiller, a bactericide agent, an epulotic agent and/or essence of eucalyptus.

According to another characteristic of the invention, the microcapsule coating is comprised of starch that has been subjected to a chemical modification to give it a positive charge, for example, via quaternization.

Another object of the invention is a process for treating fibres in a domestic washing process characterized in that after the stages of washing with detergent and rinsing of the fibres, one or more additives, separate from one another, are added, which are comprised of a microencapsulated product selected from among a cosmetic, a pharmacological drug, an evaporable product or a combination thereof, the microcapsule coating being comprised of a cationic compound.

### DETAILED DESCRIPTION OF THE INVENTION

The additive for domestic washing processes, for use once the clothes have been washed and rinsed, is designed to be incorporated in conventional, domestic washing machines, as an entity or additive separate from any fabric softener or detergent. The additive according to the invention is comprised of the desired product for impregnating the clothes, and which forms the nucleus of some microcapsules with a cationic coating. Said microcapsules are produced by the usual procedures used and known by a person skilled In the art, such as, for example, by coacervation techniques.

The additive for domestic washing processes is preferably presented in the form of a single-dose, which is placed in the washing-machine compartment used for fabric softeners or other products for the final treatment of clothes.

In addition, the additive is preferably supplied in the form of a suspension of the microcapsules in an inert liquid, such as water with an acid compound to enhance the cationic character of the microcapsule coating. In such a case, a preferred compound is one which comprises between 0.1% and 40% by weight of the microencapsulated product; between 1.0% and 20% by weight of cationic coating; and an adequate quantity for 100% by weight of water, which may comprise other dissolved compounds, such as, for example, hydrochloric acid.

The cationic coating used is a polysaccharide such as starch, or any other type of fecula, which must have a positive charge in order to be able to attach itself to the fibres of the clothing, which generally have negative charges. For example, in the case of using starch, it may have positive charges via a treatment of quaternization with dimethyl sulphate.

Among the products forming the nucleus of the microcapsules, cosmetics, pharmacological drugs or evaporable products are selected, the evaporable products being insect repellents or air fresheners.

Some examples of microencapsulated products in additives for the domestic washing process according to the invention will now be presented.

When treatment of the clothing with the additive is trying to achieve the effect of providing the fibres with the capacity to act as a transporter of a cosmetic product, the microcapsules with a coating with a cationic compound contain a moisturizing compound, such as extract of Aloe Vera; an ultra-violet ray protector, a toner, a product for reducing the effects of stress on the skin, for example, a free-radicals captor; an anti-wrinkle compound; a deodorant; a compound to slow hair-growth; or a whitening compound.

### EXAMPLE 1

### Additive With Cosmetic Product With Moisturizing Effect

60%-85% by weight of water of the total additive weight.

Microcapsules comprised of a cationic coating with a percentage weight, with respect to the total additive weight, of between 5% and 10%; and a nucleus of between 10% and 30% by weight of extract of Aloe vera and Chitosan, with respect to the total additive weight.

### EXAMPLE 2

### Additive With Cosmetic Product With Photoprotective Effect

65%-85% by weight of acidified water, of the total additive weight.

Microcapsules comprised of a cationic coating with a percentage weight, with respect to the total additive weight, of between 10% and 20%; and a nucleus of between 5% and 15% by weight of extract of Aloe Vera; Chitosan and Cinnamate, with respect to the total additive weight.

### EXAMPLE 3

### Additive With Cosmetic Product With Anti-Cellulite Effect

70%-94% by weight of water, of the total additive weight.

Microcapsules comprised of a cationic coating with a percentage weight, with respect to the total additive weight, of between 5% and 20%; and a nucleus of proliposomes, carnitine, escina, tetraethylammonium and/or caffeine, of between 1% to 10% by weight of the total additive weight.

### EXAMPLE 4

### Additive With Revitalizing, Anti-Wrinkle Cosmetic for the Skin

80%-98% by weight of acidified water, with respect to the total additive weight.

Microcapsules with a cationic coating which constitutes between 1% and 5% by weight of the total additive weight; and a nucleus which comprises extract of Aloe Vera, and/or ubiquinone and/or retinol with a percentage weight, of the total additive weight, of 1% to 5%.

### EXAMPLE 5

### Additive With Anti-Stress Cosmetic

80%-98% by weight of water of the total additive weight.

Microcapsules comprised of a cationic coating with a percentage weight of between 1% to 15%; and a nucleus which represents between 1% and 5% by weight of the total additive weight and which comprises extract of Kava (Piper methysticum) and/or serotonin.

Other examples of microencapsulated cosmetics products for administering as additives in the domestic clothes-washing process, are anti-wrinkle compounds, for example, collagenase inhibitors. These additives are specially designed to treat pillowcases.

Other additives according to the invention comprise whitening compounds or skin pigmentation inhibitors, or compounds to slow hair-growth, the latter being very suitable for washing tights which must be applied, for example, after a session of depilation. In all of these, the percentage weight of the microencapsulated product is between 1.0% and 40% of weight with respect to the total additive weight which is comprised, in addition to the microencapsulated product, of the cationic coating which surrounds the microcapsules and the liquid vehicle, such as acidified water.

Among the pharmacological products in the nucleus of microcapsules comprising the additive, bactericide or epulotic agents are also used, as are muscle relaxants. The latter are specially suited to clothing for people with muscle problems or pain.

### EXAMPLE 6

### Additive With Essence of Eucalyptus

40%-85% by weight of water, with respect to the total weight of the additive for domestic washing processes.

Microcapsules with a cationic coating, with a percentage weight with respect to the total additive weight, of between 5% and 20%; and with a nucleus of 10% to 40% by weight of the total additive weight, of extract of eucalyptus.

This additive is preferably designed for the treatment, in domestic washing machines, of sheets and other bed linen to relieve cold symptoms.

### EXAMPLE 7

### Additive With Energy Properties

80%-98% by weight of water (of the total additive weight).

Microcapsules with a cationic coating with a percentage weight, with respect to the total additive weight, of between 1% and 15%; and with a nucleus comprising essence of ginseng and moisturizers (extract of Aloe vera), with a percentage weight, with respect to the total additive weight, of between 1% and 5%.

### EXAMPLE 8

### Additive With Insect Repellent

70%-94% by weight of water, of the total additive weight.

Microcapsules, with a mosquito repellent, of between 1% to 10% by weight of the total additive weight, and with a cationic coating of between 5% and 20% by weight.

In all the examples, the cationic microcapsule coating corresponds to any type of quaternized starch, of the type known by persons skilled in the art, e.g. leguminous plants, cereal starch, etc., such as maize, rice, tapioca, which are water-soluble. Fatty acids that have been modified to give them positive charges can also be used.

The additives in the examples 1 to 8 are usually, for example, for use with underwear, socks, tights, and even sheets.

All the cosmetic, pharmacological and evaporable products are used in the form of microcapsules with the fibres of garments that, once washed and rinsed, are subjected to the action of the additive according to the invention. Once dry, the garments retain the microcapsules, with the products of interest inside them, so that, by the effect of rubbing against the skin or movement of the user, the microcapsules break and the product inside is released.

The additive object of the invention is intended for insertion in the compartment used for additives for treating clothes, once washed and rinsed, where fabric softeners are generally added. The advantage of this additive is that the user can use it separately from the fabric softener to give the clothing the desired finish. Additives according to Examples 1 to 5 can thus be used when one is seeking a continuous, long-lasting application of a certain cosmetic with the clothing that is used by the user, e.g. a moisturizer for the legs which is released while the user is wearing tights which, beforehand, were treated for this purpose.

In other cases, the user can treat sheets so that a particular fragrance or essence is evaporated on a continuous basis.

In addition, during the domestic treatment process, various additives can be applied to the garments at the same time, for example, an additive with microcapsules with a moisturizing product and an additive with microcapsules with a photoprotective product. For this, the formulation of the additive in a single-dose is sufficient for the user to make up the desired combination as required.

The additive object of the invention is designed so that it adheres between clothing fibres at the working temperatures and speeds of domestic washing machines. Additives with microcapsules can thus be used up to 60° C. without compromising their consistency and, at the same time, favouring their impregnation into the fibres. Once the additive has been applied, the garment can be dried or ironed up to a temperature range of between 140° C. and 150° C. Once the user has worn the garment treated with the additive object of the invention, it is recommended not to wash it at more than 30° C. to prolong the effect, which does not generally run out until after 4 or 5 washes.

Obviously, the additive can also be applied in cases where hand-washing of garments is preferred. In this latter case, in order to ensure that the microcapsules are incorporated uniformly and to achieve high performance (impregnation of the fibres with a higher number of microcapsules), it is recommended to leave the garment treated in the additive for at least 10 to 15 minutes, stirring from time to time.

The additive for domestic washing processes, in addition to using an additive with a formulation that is not complex, leads to positive results deriving not solely from the cosmetic or pharmacological effect it has, but also in terms of its long-lasting effect. In this way, it should be highlighted that, with a treatment of this type, the garments manage to exert the desired effect, whether they are moisturizing or photoprotective products, an insecticide, painkiller, etc., at least for four washes without subsequent treatment with additives.

The additive for domestic washing processes, object of the invention, may be added in the form of a fine powder, the microcapsules having a granular size of between 5 μm and 15 μm, which is also the size of the microcapsules when they are applied in suspension in a liquid vehicle. Once the clothing has been washed and rinsed, the same water that enters the washing-machine compartment used to put all additives for special treatment of garments, is responsible for incorporating the powder particles or microcapsules therein, making up the liquid medium that will come into contact with the fibres. This allows for a more uniform distribution of the additive in all the clothes.

1. Additive for domestic washing processes, for incorporation in the process after washing and rinsing, characterized in that it is comprised of a microencapsulated product which is either a cosmetic, a pharmacological drug, an evaporable product or a combination thereof, the microcapsule coating being comprised of a cationic compound.

2. Additive according to claim 1, characterized in that it is in the form of a single-dose.

3. Additive according to claims 1 or 2, characterized in that it is in the form of a liquid.

4. Additive according to any of claims 1 to 3, characterized in that the cosmetic product is taken separately from among a moisturizing compound, an ultra-violet ray protector, a toner, a product for reducing the effects of stress on the skin, an anti-wrinkle compound, a deodorant, a compound to slow hair-growth and/or a whitening compound.

5. Additive according to any of claims 1 to 3, characterized in that the pharmacological product is selected separately from among a painkiller, a bactericide agent, an epulotic agent and/or essence of eucalyptus.

6. Additive according to any of claims 1 to 3, characterized in that the microcapsule coating is comprised of quaternized starches.

7. Process for treating fibres in a domestic washing process, characterized In that after the stages of washing with detergent and rinsing of the fibres, one or more additives, separate from one another, are added, which are comprised of a microencapsulated product selected from a cosmetic, a pharmacological drug, an evaporable product or a combination thereof, the microcapsule coating being comprised of a cationic compound.
